(19) United States
(12) Patent Application Publication
(10) Pub. No.: US 2012/0191012 A1
(43) Pub. Date: Jul. 26, 2012

(54) Controllable Stiffness Guidewire

(75) Inventors: Albert K. Chin, Palo Alto, CA (US); Lishan Aklog, Scottsdale, AZ (US)

(73) Assignee: Pavilion Medical Innovations

(21) Appl. No.: 13/357,043

(22) Filed: Jan. 24, 2012

Related U.S. Application Data
(60) Provisional application No. 61/435,517, filed on Jan. 24, 2011.

(51) Int. Cl. A61M 25/09

(52) U.S. Cl. 600/585

(57) ABSTRACT

Controllable stiffness guidewires and methods of using such guidewires are disclosed. According to aspects illustrated herein, there is provided a controllable stiffness guidewire that includes a substantially flexible core wire having a distal section and a proximal section. A plurality of beads may be slidably disposed between the distal section and the proximal section of the core wire. In an embodiment, the beads may be contiguous with one another. The guidewire may further include an actuator designed to compress the beads against one another along the core wire. By compressing the beads against one another, the stiffness of the core wire, and thus the guidewire, can be variably increased between substantially flexible and substantially rigid.

### RELATED APPLICATIONS

This application claims the benefit of and priority to U.S. Provisional Patent Application No. 61/435,517, filed on Jan. 24, 2011, the entirety of which is hereby incorporated herein by reference for the teachings therein.

### FIELD

The presently disclosed embodiments relate to guidewires, and more particularly to guidewires with controllable stiffness and methods of using such guidewires.

### BACKGROUND

Minimally-invasive surgical techniques, including techniques for treatment of cardiovascular conditions, are becoming increasingly popular. For example, angioplasty, which is a minimally-invasive surgical technique for treating stenosis where there is an abnormal restriction in a blood vessel, has become a common choice over more invasive procedures open heart surgery. In angioplasty, a balloon catheter may be advanced through the vasculature into a restricted area in a coronary artery. The balloon may then be expanded against the restricted area to open the artery for increased blood flow. Subsequently, a stent may be placed in the artery to keep the artery open over time.

Guidewires are typically employed to provide a path over which the catheter may be advanced through the vasculature to the site of stenosis. In a typical guidewire, the rigidity decreases progressively from the proximal tip to the distal tip, with the distal tip being substantially flexible. With a substantial segment of a typical guidewire being substantially flexible, it can be difficult to facilitate advancement of the guidewire through the vasculature to the site of stenosis. In addition, once the guidewire is positioned in the desired location, the flexibility of the guidewire can be problematic. For one, a guidewire that is too flexible may not be able to provide sufficient pathway to support the catheter as the catheter advances toward the site of stenosis. Such a flexible guidewire is also prone to being pulled out or displaced from the blood vessel, when a relatively stiff therapeutic catheter is advanced over the guide wire, or due to recoil forces on the catheter, such as when inflating a balloon or delivering a stent. To re-position the guidewire, the catheter may need to be removed, which can be extremely frustrating to the surgeon, and may also increase the duration and cost of the procedure at the expense of the patient.

Therefore, there is a need for a guidewire with a controllable stiffness.

### SUMMARY

According to aspects illustrated herein, there is provided a controllable stiffness guidewire that includes a substantially flexible core wire having a distal section and a proximal section. A plurality of beads may be slidably disposed between the distal section and the proximal section of the core wire. In an embodiment, the beads may be contiguous with one another. The guidewire may further include an actuator designed to compress the beads against one another. By compressing the beads against one another, the stiffness of the core wire, and thus the guidewire, can be variably increased between substantially flexible and substantially rigid.

According to aspects illustrated herein, there is further provided a controllable stiffness guidewire that includes a sleeve. The guidewire may further comprise a substantially flexible core wire positioned within the sleeve. A fluid may also be provided within the sleeve, such that removal of the fluid from within the sleeve causes the sleeve to collapse around the core wire to variably increase the stiffness of the core wire, and thus the guidewire, between substantially flexible and substantially rigid.

According to aspects illustrated herein, there is also provided a method of delivering a catheter to a site of interest. Initially, a guidewire having a substantially flexible core wire and a plurality of contiguous beads slidably disposed between a distal section and a proximal section of the core wire may be advanced to the site of interest. Once the guidewire is at the site of interest, the beads may be compressed along the core wire against one another to form a track of a preferred rigidity. Next, a catheter may be directed over the track to the site of interest.

According to aspects illustrated herein, there is further provided another method of delivering a catheter to a site of interest. Initially, a guidewire comprising a sleeve containing a fluid and a substantially flexible core wire positioned within the sleeve may be advanced to the site of interest. Once the guidewire is at the site of interest, an amount of fluid may be removed from within the sleeve to form a track of a preferred rigidity. Next, a catheter may be directed over the track to the site of interest.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1A is a schematic view of a controllable stiffness guidewire of the present disclosure.

FIG. 1B is a cross-sectional view of a bead of a controllable stiffness guidewire of the present disclosure.

FIGS. 2A-2D illustrate various embodiments of the controllable stiffness guidewire of FIG. 1.

FIGS. 3A-3B illustrate an embodiment of an actuator suitable for use in connection with the controllable stiffness guidewire of FIG. 1.

FIGS. 4A-4C illustrate another embodiment of an actuator suitable for use in connection with the controllable stiffness guidewire of FIG. 1.

FIGS. 5A-5B illustrate an alternative embodiment of a controllable stiffness guidewire of the present disclosure.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

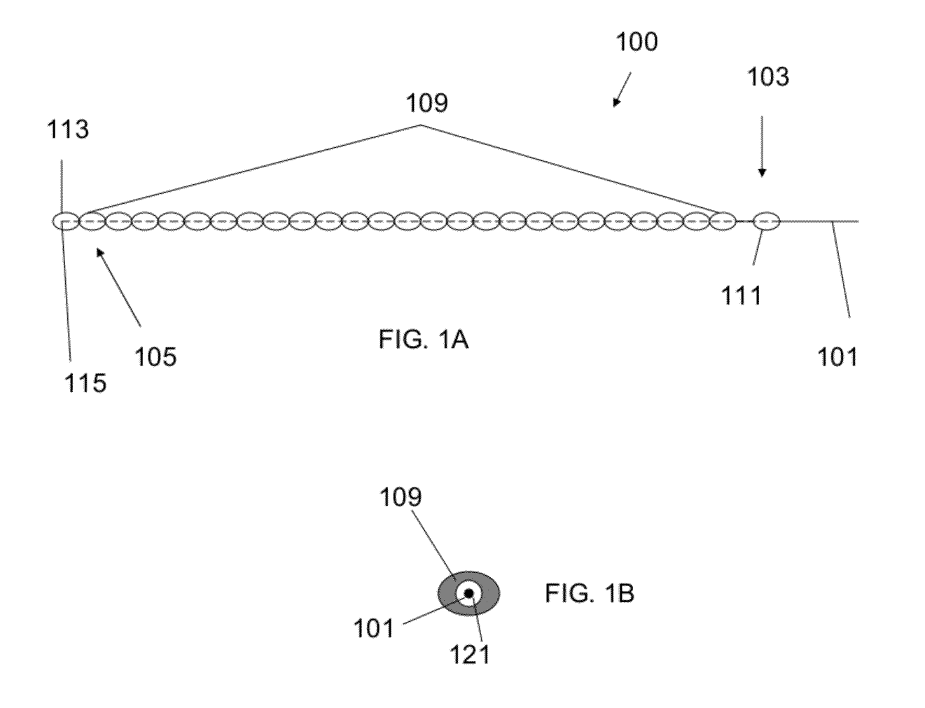

A guidewire 100 in accordance with an embodiment of the present disclosure is shown generally in FIG. 1A. The guidewire 100 may include a core wire 101 having a proximal section 103, a distal section 105, and a longitudinal axis therebetween. The core wire 101, in an embodiment, may be formed from any relatively flexible, conventional guidewire material, such as stainless steel, titanium, nickel-titanium alloy, tantalum, alloys thereof, or combinations thereof. In one embodiment, the outer diameter and rigidity of the core wire 101 may increase progressively from the distal section 105 toward the proximal section 103 of the core wire 101 to aid in the advancement of the guidewire 100 through the vasculature.

The guidewire 100 may also include a plurality of beads 109 disposed along a length of the core wire 101, for instance, between the distal section 105 and the proximal section 103 of the core wire 101. The beads 109 may be employed to impart a desired or preferred rigidity to the guidewire. As illustrated in FIG. 1A, in an embodiment, the beads 109 may be contiguous with one another, that is, there may be substantially no gap between the adjacent beads 109.

The term “bead” as used herein refers to an object of any shape, design or construction that can be pierced for threading or stringing along the core wire 101. In an embodiment, the beads are sized such that the guidewire 100 is appropriately sized for the procedure to be performed using the guidewire 100. The beads 109 may thus be of various shapes, including, but not limited to, spherical, oval, tubular, ring-shaped, disc-shaped, barrel-shaped, bean-shaped, or barbell-shaped or any combination thereof. In an embodiment, adjacent beads may have a similar shape or maybe of different shape from one another. Likewise, adjacent beads may be of a similar size or of different size than one another.

In an embodiment, the beads 109 may have a smooth rounded outer surface to minimize friction between the beads 109 and the inner surface of a vessel along which the guidewire 100 is being advanced. To further minimize the friction, the beads 109 may be made of a material, such as PTFE, that can minimize or reduce friction or may be coated with a hydrophilic coating, such as, for example, polyvinylpyrrolidone, polyurethane, poly(acrylic acid), poly(methacrylic acid), poly(dimeth)acrylamide, PTFE, poly(acrylamide), polyvinybutyrol, poly(hydroxyethylmethacrylate) or combinations thereof. Additionally or alternatively, the beads 109 may be coated with an anti-thrombogenic, such as heparin (or its derivatives), urokinase, or PPack (dextrophenylalanine proline arginine chloromethylketone) to prevent thrombosis or any other adverse reaction due to the introduction of the guide wire into a body of a patient. To minimize friction of the beads 109 along the guidewire 100, the core wire 101 may also be made of a material that can minimize or reduce friction. Alternatively, the core wire 101 may be coated with a hydrophilic coating to reduce friction between the core wire 101 and the beads 109 and/or a vessel along which the guidewire 100 is being advanced.

Each bead 109, in one embodiment, may include a bore 121 through which the core wire 101 can extend, as illustrated in FIG. 1B. The bore 121 may, in an embodiment, be placed substantially through the center of the bead 109. Of course, should it be desired, the bore 121 may be placed off-center of the bead 109. In an embodiment, the bore 121 may have a diameter larger than that of the core wire 101, so as to allow the beads 109 to be threaded over the core wire 101 and to slide along the core wire 101. To retain the beads 109 on the core wire 101, the core wire 101 may include a distal retainer 113, a proximal retainer 111, or both, fixedly disposed on the core wire 101. In an embodiment, the distal retainer 113 may be employed to limit the range of motion of the beads 109 in the distal direction. To achieve this, the distal retainer 113 may be attached to the core wire 101 distally of the plurality of beads 119. The distal retainer 113 may, in various embodiments, have a similar or different size and shape as the beads 109. In an embodiment, it may be desirable to provide the core wire 101 with a blunt distal tip to avoid puncturing or otherwise damaging blood vessels. For that reason, in an embodiment, the distal retainer 113 may extend past a distal terminal tip 115 of the core wire to enclose the distal terminal tip 115 of the core wire 101. In an embodiment, the distal retainer 113 may be rounded in order to provide the core wire 101 with a blunt distal tip. Similarly, in an embodiment, the proximal retainer 111 may be employed to limit the range of motion of the beads 109 in the proximal direction, and thus may be disposed proximally of the plurality of beads 119. The proximal retainer 111 may, in various embodiments, have a similar or different size and shape as the beads 109. In an embodiment, the most proximal bead of the plurality of beads 109 may serve as the proximal retainer 111. Similarly, the most distal bead of the plurality of beads 109 may serve as the distal retainer 113.

When the beads 109 are in their initial position, referred to herein as a non-compressed or relaxed position, the flexibility of the guide wire 100 may be maximized. Alternatively, when the beads 109 are compressed against one another, the beads 109 together may impart a controllable stiffness to the guidewire 100, corresponding to the degree of compression of the beads 109. Specifically, increasing compression of the beads 109 may increase the rigidity of the guidewire 100, whereas reducing compression of the beads 109 may increase the flexibility of the guidewire 100. Accordingly, the level of rigidity of the guidewire may be varied between substantially fully flexible, when the beads are in the relaxed position, to substantially rigid, when the beads are in the fully compressed position. The guidewire 100, as provided, can be advanced along multiple curves that naturally occur in the vasculature to define a 3-dimensional pathway or track, which, can be imparted with a preferred rigidity by compressing the beads 109 against one another, to form a stationary reference track for advancement of, for example, a catheter, to a desired location in the vasculature. It should be noted that the beads 109 can be compressed against one another by either pushing the beads 109 distally against the distal retainer 113 or by tensioning the core wire 101 by pulling the core wire 101 proximally while holding the beads 109 in place.

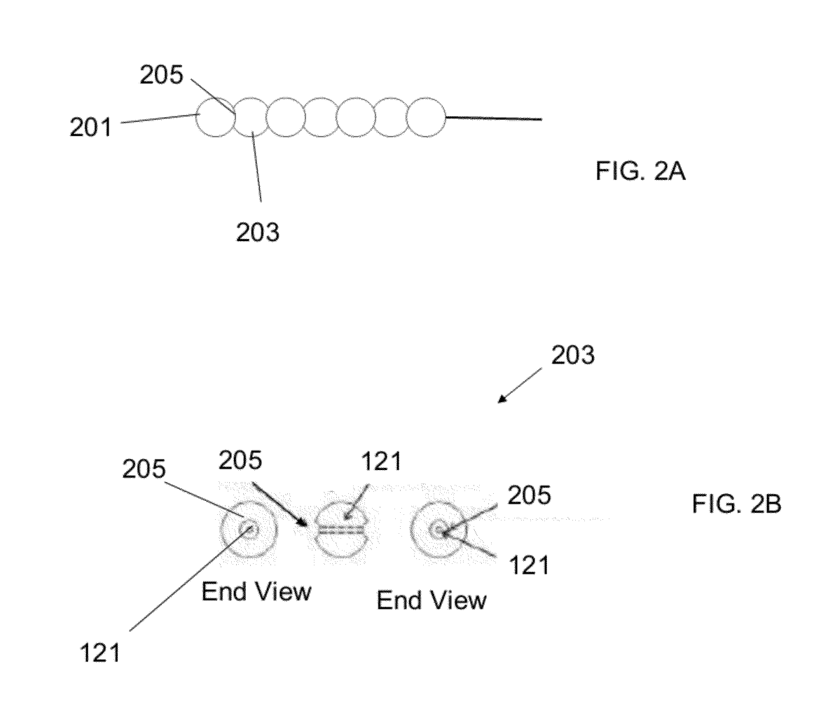

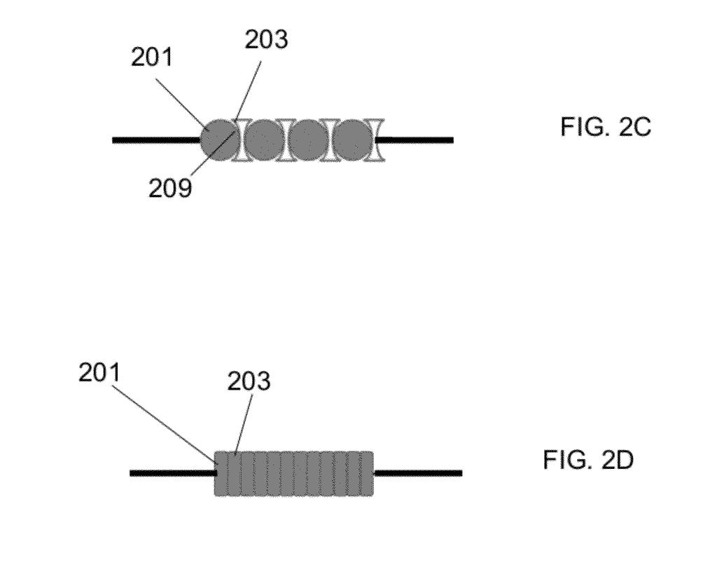

In some embodiments, as illustrated in FIGS. 2A-2D, the plurality of beads 109 may be configured to enhance an area of contact between adjacent beads. Increasing the area of contact between the adjacent beads can increase the friction between the adjacent beads, when tension is applied to the core wire 101, thus potentially increasing the rigidity of the guidewire 100. In an embodiment, complimentary shaped beads may be alternated to impart a desired rigidity. The complimentary shapes may be selected so that, when the beads 109 are compressed against one another, the adjacent beads may become mated or substantially flush with one another. For example, as illustrated in FIG. 2A, between any two adjacent beads 201, 203, the fist bead 201 may be of a substantially spherical shape, while the second bead 203, adjacent to the first bead 201, may include a countersink 205 to permit a substantially flush engagement with the first bead 201. FIG. 2B is a close-up of a bead 203 having a countersink 205. In an embodiment, the countersink 205 may define a concave cavity that can complementary receive a portion of the first bead 201.

In another embodiment, as illustrated in FIG. 2C, the first bead 201 may again be of a substantially spherical shape, while the second bead 203 may be disc-shaped with a concave surface. The concave surface of the second bead 203 may define a cavity 209 that can complementary receive a portion of the first bead 201. Accordingly, in such embodiments, when the beads 109 are compressed, the first bead 201 can be pushed into the cavities 205, 209 of the second bead, thus increasing the area of contact between the adjacent beads. Alternatively, to increase the area of contact between adjacent beads 201, 203, all beads may be shaped so the contacting sides of the beads 201, 203 may come flush against the contacting sides of adjacent beads when the beads 109 are compressed, as shown in FIG. 2D. To achieve this goal, in an embodiment, at lest some of the beads 109 may have flat sides parallel to one another.

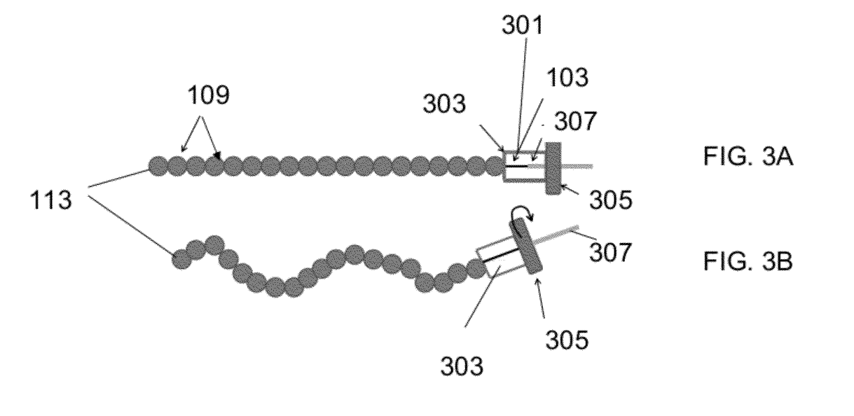

Referring now to FIGS. 3A and 3B, an actuator 301 for compressing the beads 109 against one another may be provided. The actuator 301 may, in an embodiment, be positioned at the proximal section 103 of the core wire 101. The actuator 301 can act to releasably compress the beads 109 against one another, so as to vary the rigidity of the guidewire 100. In an embodiment, as illustrated in FIG. 3A, the actuator 301 may comprise a frame 303, a knob 305 affixed to the frame 303, and a threaded member 307 attached to the proximal section 103 of the core wire 101 and received through a threaded bore 309 in the knob 307. The threaded member 307 may be moved by rotating the knob 305, which also moves the frame 303 along the core wire 101. As illustrated in FIG. 3B, rotating the knob 305 to slide the frame 303 distally along the core wire 101 may compress the beads against 109 each other to increase the rigidity of the guide wire 100. On the other hand, rotating the knob 305 to slide the frame 303 proximally along the core wire 101 may allow the beads 109 to return toward their original positions, thus decreasing the rigidity of the guidewire 100, as illustrated in FIG. 3A.

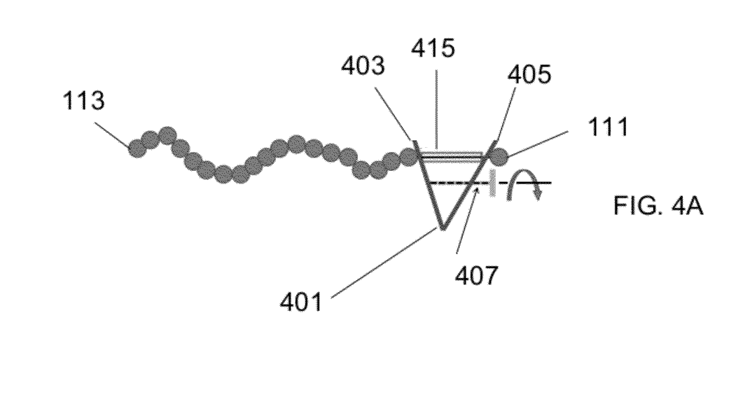

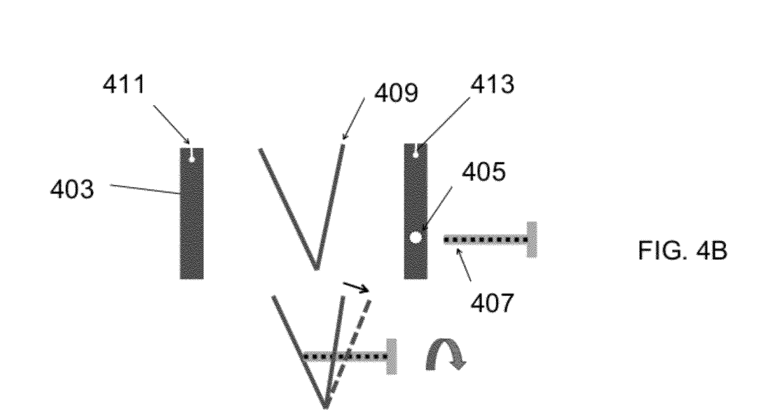

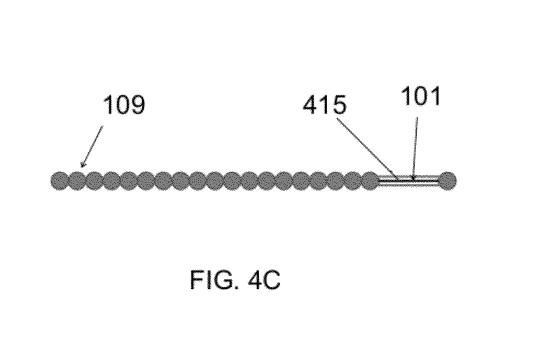

In another embodiment, as illustrated in FIG. 4A, the actuator 301 may comprise a v-shaped frame 401 having a first arm 403 and a second arm 405 positioned at the proximal section 103 of the core wire 101. An adjustment screw 407 may also be provided for adjusting the position of the first arm 403 relative to the second arm 405. Referring to FIG. 4B, to form the v-shaped frame 401, the first and second arms 403, 405 may be disposed over a spring form 409, which may bias the arms toward each other.

To use such an actuator, the v-shaped frame 401 may be placed along the core wire 101 at the proximal section 103 of the core wire 101. In an embodiment, there may be a gap between the proximal retainer 111 and the most proximal bead 109 to accommodate the actuator 301. Alternatively, the actuator 301 itself can be utilized to prevent the beads 109 from sliding off the proximal section 103 of the core wire 101. To that end, the first arm 403 and the second arm 405 may include slots 411 and 413, respectively, for receiving the core wire 101 therethrough. Once the v-shaped frame 401 is placed onto the core wire 101, the extent of compression of the beads 109, and thus the rigidity of the guidewire 100, may be controlled with the adjusting screw 407.

In an embodiment, the actuator 113 can be removably attached to the core wire 101. In an embodiment, as illustrated in FIG. 4C, an elastic spacer 415 may be provided along the core wire 101 between the proximal retainer 111 and the most proximal bead. The elastic spacer 415 may keep the beads 109 contiguous with respect to one other as the guide wire 100 is navigated to the site of interest. In an embodiment, the elastic spacer 415 may not affect the flexibility of the guidewire 100, that is, the beads 119 may remain in their original position to maximize the flexibility of the guidewire 100 even when the elastic spacer 415 is engaged with the core wire 101. The v-shaped frame 401 may be inserted onto the guidewire 100 about the elastic spacer 415, when the rigidity of the guidewire 100 needs to be varied.

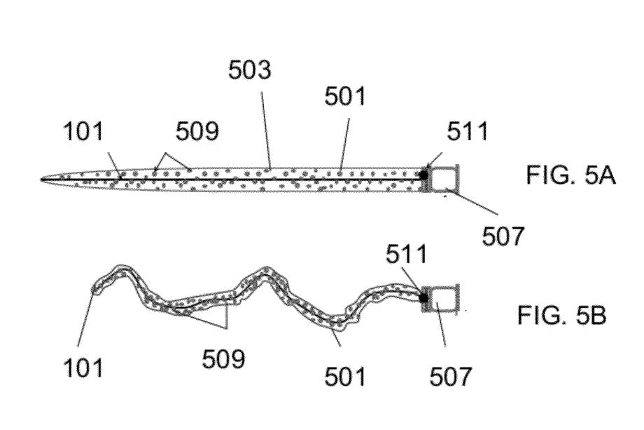

FIG. 5A and FIG. 5B illustrate another embodiment of a controllable stiffness guidewire 100 of the present disclosure. In such an embodiment, the core wire 101 may extend within a sleeve 501. In an embodiment, the sleeve 501 may extend between the distal section 105 and the proximal section 103 of the core wire 101. The sleeve 501, of course, may be designed to extend only along a portion of the core wire 101, should that be desired. The rigidity of the guidewire 100, in this embodiment, may be varied by varying the amount of fluid 503, i.e., gas or liquid, in the sleeve 501. However, the sleeve 501 may be filled with fluid 503 to maximize the flexibility of the guidewire 100, as illustrated in FIG. 5A. To accommodate fluid 503, the sleeve 501 may be made with a substantially impermeable biocompatible material. In an embodiment, the sleeve 501 may be sealed around the core wire 101. The sleeve 501 may be provided with an opening 511, through which fluid 501 may be added to or removed from the sleeve 501. By removing the fluid 511 from within the sleeve 501, the sleeve 501 can be collapsed around the core wire 101 to form a rigid structure, as illustrated in FIG. 5B. To further increase the rigidity of the guidewire 100, a plurality of particles 509, such as, for examples, microspheres, may be contained within the sleeve 501. In the presence of particles 509, when the fluid 503 is withdrawn from the sleeve 501, the plurality of particles 509 may be compressed between the core wire 101 and the sleeve 501 to further increase the rigidity of the guidewire 100. In an embodiment, a filter 507 may be provided to ensure that the particles 509 are not removed from the sleeve 501 with the fluid 503.

In operation, the guidewire 100 may be used to provide a track for delivering a catheter to a desired location in the vasculature. To gain access to the vasculature, in an embodiment, a needle may first be used to provide an opening in a blood vessel, typically the femoral artery, through which the guide wire 100 may be inserted into the blood vessel. Initially, the beads 109 may be positioned in the relaxed position along the core wire 101 to maximize the flexibility of the guidewire 100. Alternatively, the beads 109 may be compressed to a desired extent, so as to impart a desired rigidity to the guidewire 100. The guidewire 100 may then be advanced to the desired location through the vasculature by manipulating the proximal section 103 of the guidewire 100. In an embodiment, to facilitate manipulation of the guidewire 100 a torque device may be provided for attachment to the proximal section 103 of the guidewire 100. As the guidewire 100 moves to the desired location, the rigidity of the guidewire 100 may be varied as needed to negotiate the turns and curves of the vasculature.

Once the guidewire 100 is positioned at the desired location, the beads 109 may be compressed, as needed, to impart desired rigidity to the guidewire 100. The guidewire 100 may thus form a track of desired rigidity over which a catheter can be advanced to the desired location. In an embodiment, the guidewire 100 may be employed as an anchor point from which passage of a catheter through an area of resistance may be performed with increased force.

By way of a non-limiting example, the guidewire 100 may be used with a catheter system for bypassing, or minimizing resistance across, an obstruction or area of critical stenosis or tortuosity, such as a clot, within a vessel. One suitable catheter system for bypassing, or minimizing resistance across, an obstruction is disclosed in co-pending U.S. application Ser. No. 13/267,657, the entirety of which is hereby incorporated herein by reference for the teachings therein. Briefly, a catheter system for bypassing, or minimizing resistance across, an obstruction or area of critical stenosis or tortuosity may include, in one embodiment, a sleeve having a distal end, a proximal end, and a pathway therebetween. The distal end of the sleeve, in an embodiment, may be used to extend across an obstruction and bypass the obstruction. In an embodiment, the system may further include a balloon having a proximal end, a close-ended distal end, and a lumen therebetween. The distal end of the balloon may be designed to move from an inverted position where the distal end is positioned within the lumen of the balloon, to an everted position where the distal end of the balloon is capable of delivering the sleeve across the obstruction.

In operation, the catheter may be advanced immediately proximal to a site of interest in a blood vessel, such as, the stenosis, occlusion, or area of tortuosity. Pressurization of the inverted balloon may causes the balloon and the sleeve within the balloon to evert through the stenosis, occlusion or tortuosity. As the balloon is inflated to evert itself and the sleeve residing inside it, the everting end of the balloon/sleeve may contact the stenosis or occlusion. If the stenosis is very tight, or if an occlusion exists, a backforce may develop upon balloon inflation that may tend to push the catheter backwards along the blood vessel and even out of the blood vessel.

The guidewire 100 of the instant disclosure may be employed to counteract such backforce on the catheter to ensure that the catheter stays in place as the balloon is everted through the stenosis, occlusion or tortuosity. To that end, the guidewire, in its flexible state, may be first advanced to the site of interest, as described above. Once at the site of interest, the beads 109 may be compressed as needed to impart the desired rigidity to the guidewire 100. The guidewire 100 may thus form a track over which the catheter may be advanced to the site of interest and may serve as an anchor point for evertion of the balloon.

In an embodiment, a controllable stiffness guidewire may include a substantially flexible core wire having a distal section and a proximal section. A plurality of beads may be slidably disposed between the distal section and the proximal section of the core wire. In an embodiment, the beads may be contiguous with one another. The guidewire may further include an actuator designed to compress the beads against one another. By compressing the beads against one another along the core wire, the stiffness of the core wire, and thus the guidewire, can be variably increased between substantially flexible and substantially rigid.

In an embodiment, a controllable stiffness guidewire may includes a sleeve. The guidewire may further comprise a substantially flexible core wire positioned within the sleeve. A fluid may also be provided within the sleeve, such that removal of the fluid from within the sleeve causes the sleeve to collapse around the core wire to variably increase the stiffness of the core wire, and thus the guidewire, between substantially flexible and substantially rigid.

In an embodiment, a method of delivering a catheter to a site of interest may include an initial step of advancing a guidewire having a substantially flexible core wire and a plurality of contiguous beads slidably disposed between a distal section and a proximal section of the core wire to the site of interest. Once the guidewire is at the site of interest, the beads may be compressed along the core wire against one another to form a track of a preferred rigidity. Next, a catheter may be directed over the track to the site of interest.

In an embodiment, a method of delivering a catheter to a site of interest may include an initial step of advancing a guidewire comprising a sleeve containing a fluid and a substantially flexible core wire positioned within the sleeve to the site of interest. Once the guidewire is at the site of interest, an amount of fluid may be removed from within the sleeve to form a track of a preferred rigidity. Next, a catheter may be directed over the track to the site of interest.

All patents, patent applications, and published references cited herein are hereby incorporated by reference in their entirety. While the invention has been described in connection with the specific embodiments thereof, it will be understood that it is capable of further modification. It will be appreciated that several of the above-disclosed and other features and functions, or alternatives thereof, may be desirably combined into many other different systems or applications. Furthermore, this application is intended to cover any variations, uses, or adaptations of the invention, including such departures from the present disclosure as come within known or customary practice in the art to which the invention pertains, and as fall within the scope of the appended claims.

What is claimed is:

1. A guidewire comprising:
a substantially flexible core wire having a proximal section and a distal section;
a plurality of beads slidably disposed between the distal section and the proximal section of the core wire, and positioned in a contiguous relationship with one another; and
an actuator, designed to compress the beads against one another, so as to variably increase the stiffness of the guidewire between a substantially flexible and a substantially rigid state.

2. The guidewire of claim 1, further comprising a distal retainer at the distal section of the core wire such that the plurality of beads are compressed against the distal retainer to increase the stiffens of the guidewire.

3. The guidewire of claim 1, wherein the plurality of beads includes alternating complimentary-shaped beads, such that, when the alternating complimentary-shaped beads are compressed against one another, the alternating complimentary-shaped beads become mated with one another.

4. The guidewire of claim 1, wherein the plurality of beads includes a first bead and an adjacent second bead, each the first bead and the second bead having a substantially flat contacting side, such that, when the first bead and the second bead are compressed against one another, the contacting side of the first bead and the contacting side of the second bead come substantially flush against one another.

5. A guidewire comprising:
a sleeve;
a substantially flexible core wire positioned within the sleeve;
a fluid contained within the sleeve, such that removal of the fluid from within the sleeve causes the sleeve to collapse around the core wire to variably increase the stiffness of the core wire, and thus the guidewire, between substantially flexible and substantially rigid.

6. The guidewire of claim 5 wherein the substantially flexible core wire is positioned within the sleeve in its entirety.

7. The guidewire of claim 5 further comprising a plurality of particles disposed within the sleeve to be compressed against the substantially flexible core wire when fluid is removed from the sleeve.

8. A method of delivering a catheter to a site of interest, the method comprising:
advancing a guidewire having a substantially flexible core wire and a plurality of contiguous beads slidably disposed between a distal section and a proximal section of the core wire to a site of interest;
compressing the beads along the core wire against one another to form a track of a preferred rigidity; and
directing a catheter over the track to the site of interest.

9. The method of claim 8, wherein in the step of advancing, the beads include alternating complimentary-shaped beads, such that, when the alternating complimentary-shaped beads are compressed against one another, the alternating complimentary-shaped beads become mated with one another.

10. The method of claim 8, wherein in the step of advancing, the beads include a first bead and an adjacent second bead, each the first bead and the second bead having a substantially flat contacting side, such that, when the first bead and the second bead are compressed against one another, the contacting side of the first bead and the contacting side of the second bead come substantially flush against one another.

11. The method of claim 8 wherein in the step of compressing, the beads are compressed against a distal retainer at the distal section of the core wire.

12. A method of delivering a catheter to a site of interest, the method comprising:
advancing a guidewire comprising a sleeve containing a fluid and a substantially flexible core wire positioned within the sleeve to a site of interest;
removing an amount of the fluid from within the sleeve to form a track of a preferred rigidity; and
directing a catheter over the track to the site of interest.

13. The method of claim 12 wherein in the step of advancing, the substantially flexible core wire is positioned within the sleeve in its entirety.

14. The method of claim 12 wherein in the step of advancing, the guidewire further includes a plurality of particles disposed within the sleeve to be compressed against the substantially flexible core wire when fluid is removed from the sleeve.
